(19) United States
(12) Patent Application Publication
(10) Pub. No.: US 2008/0146989 A1
(43) Pub. Date: Jun. 19, 2008

(54) Anti-repulsion anti-clogging system and method for driving a dual-axis lensectomy probe

(76) Inventor: Jaime Zacharias, Santiago (CL)

Correspondence Address: JAIME ZACHARIAS, AV. LUIS PASTEUR 5917 - VITACURA, SANTIAGO, 6670775, omitted

(21) Appl. No.: 11/754,285

(22) Filed: May 26, 2007

Related U.S. Application Data
(60) Provisional application No. 60/820,792, filed on Jul. 30, 2006.

(51) Int. Cl. A61N 7/00

(52) U.S. Cl. 604/22

(57) ABSTRACT

An anti-repulsion anti-clogging system for a dual axis lensectomy handpiece consisting in power modulation of bursts of axial oscillatory activity intercalated between bursts of rotational oscillatory activity. Power modulation of the axial bursts comprises reduction of the rise speed of the attack portion of the envelope below 0.75 mils per millisecond. Alternatively power modulation can be in the form of intercalation of a train of pulses of axial oscillations between the bursts of rotational oscillatory activity. The pulses composing the intercalated train can be of equal or different amplitudes. Repulsion of lens fragments is significantly reduced by power modulating the axial pulses intercalated between the rotational pulses as described in the present invention.

### CROSS REFERENCE TO RELATED APPLICATIONS

This application makes reference to provisional patent application No. 60/820,792 filed Jul. 30, 2006

### FIELD OF THE INVENTION

This invention relates to ultrasonic removal of the lens of the eye and more particularly to a method for driving an ultrasonic dual-axis lensectomy handpiece.

### DISCLOSURE OF PRIOR ART

Until recently, the preferred method for ultrasonic removal of the cataractous lens had considered producing axial oscillations of a hollow lensectomy probe using an electro-mechanic transducer typically of piezoelectric nature. A recent advance in the field has been the introduction of a twin-axial lensectomy system that can produce rotational ultrasonic oscillations of the lensectomy probe as well as the conventional axial oscillations.

Current twin-axial systems can produce rotational and axial oscillations only in an interleaved manner because design limitations impede having both oscillation patterns occurring simultaneously. Among the advantages of incorporating the rotatory oscillatory motion is a reduced risk of surgical wound thermal injury, reduced chatter and reduced repulsion of lens fragments, reduced turbulence in the anterior chamber of the eye, reduced acoustic cavitation and improved followability. The preferred lensectomy probes to use with the rotational system are typically funnel shaped having a wide distal opening followed by a narrowing of the axial diameter downstream toward the fixed proximal end at the surgical handpiece body. This probe design is preferred because it is more efficient to grasp and remove lens fragments while protecting from potentially dangerous post-occlusion surges in the fluid chambers of the eye. The most typical tip shapes with narrowing lumen toward the proximal end are the flared and the taper shaped tips. A known limitation of the currently available systems that incorporate ultrasonic rotational motion at the distal end of the lensectomy tip is that when used with funnel shaped tips, such as the flared and tapered tips, there is an increase in the chance of clogging of the narrowing fluidic path of the lensectomy probe by harder or leathery fragments of the cataract being removed.

Clogging is typically noticed by the surgeon because the clear fluid in the anterior chamber of the eye becomes milky and also because the lensectomy console alerts of sustained vacuum. Clogging of the lensectomy probe increases the risk of post-occlusion surge, of wound temperature rise, and produces unwanted delay in the surgical procedure, all aspects that promote surgical complications. To solve the problem of frequent clogging that is characteristic of the rotational motion of the tip of the lensectomy probe, current systems allow an operator to program the delivery of bursts of axial ultrasonic motion interleaved with the preferred rotational ultrasonic motion bursts. These axial bursts are effective to unclog the tip. Unfortunately, the unclogging action of the axial bursts of ultrasonic motion of the lensectomy tip is accompanied by increased repulsion of the lens fragments, kicking them away from the aspiration opening. The repulsive action of axial ultrasound results in unwanted chatter, turbulence, loss of efficiency, and in an increased exposure of the corneal endothelium to impacts from lens fragments spinning inside the anterior chamber of the eye.

### OBJECTS AND ADVANTAGES

It is an object of the present invention to provide a methods to operate ultrasonic lensectomy probes to prevent and resolve unwanted clogging. This method reduces lens fragment repulsion, fragment chatter, fluid turbulence in the anterior chamber, post-occlusion surge and acoustic cavitation when using twin-axial capable lensectomy systems.

### BRIEF DESCRIPTION OF THE DRAWINGS

FIG. 1 is a block diagram of an embodiment of the ultrasonic driver used to implement the method of the present invention for a twin-axial ultrasonic lensectomy handpiece.

FIG. 2 depicts a dual-trace scope recording of a system using the driving method of the prior art. An upper tracing corresponds to the driver output voltage and a lower tracing corresponds to the axial and rotational motion produced.

FIG. 3 depicts a dual-trace scope recording of a system using one embodiment of the driving method of the present invention. An upper tracing corresponds to the driver output voltage and a lower tracing corresponds to the axial and rotational motion produced.

FIG. 4 depicts a dual-trace scope recording of a system using one embodiment of the driving method of the present invention. An upper tracing corresponds to the driver output voltage and a lower tracing corresponds to the axial and rotational motion produced.

FIG. 5 depicts a dual-trace scope recording of a system using one embodiment of the driving method of the present invention. An upper tracing corresponds to the driver output voltage and a lower tracing corresponds to the axial and rotational motion produced.

### LIST OF REFERENCE NUMERALS

Ultrasonic driver subsystem 8, microcontroller 10, connector 12, external control bus 14, connector 16, programmable clock generator 18, connector 20, actuator power generator 22, connector 23, step-up transformer 24, connector 26, electro-mechanic actuator 28, nosecone 30, nosecone groove 31, lensectomy probe 32, probe distal end 34, handpiece enclosure 35, aspiration connector 36, actuator current detector 39, connector 40, actuator voltage detector 41, connector 42, connector 50, digital-to-analog converter 54, connector 56, boost dc-dc converter 58, connector 59, programmable power supply 100.

### SUMMARY

A method of driving a lensectomy probe to reduce lens fragment repulsion and chatter during the operation of twin-axial capable lensectomy systems comprising the delivery of slowly rising bursts of axial oscillations and/or trains of axial oscillations to the lensectomy needle.

### DESCRIPTION

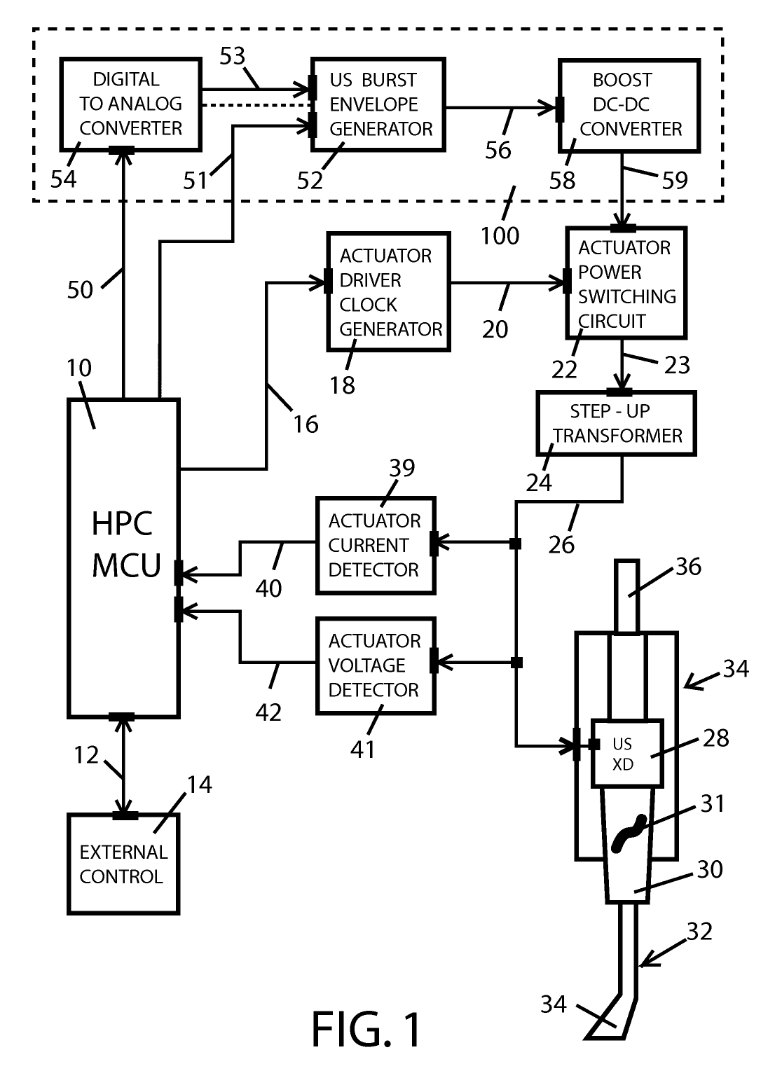

As shown in FIG. 1 the ultrasonic probe driving method of the present invention for a twin-axial lensectomy handpiece 34 typically includes a microcontroller unit 10 receiving operational settings through a connector 12 from an external controller 14 such as a lensectomy console host system. Microcontroller 10 provides an output clock signal through connector 16 for an ultrasonic actuator driver clock generator 18. Clock generator 18 can produce a range of selected frequencies through connector 20 for an ultrasonic actuator power switching circuit 22 composed by power MOSFETS such as IRF540. Microcontroller 10 also provides an output power signal through connector 50 for a digital-to-analog converter 54 such as LTC1329. Digital-to-analog converter 54 produces a voltage/current output signal. The output signal of digital-to-analog converter 54 can be fed through connector 53 to an ultrasonic burst envelope generator circuit 52. The output of circuit 52 is provided through connector 56 to a boost DC-DC converter circuit 58. Alternatively, connector 53 can skip circuit 52 and continue directly as connector 56 to directly deliver the output of analog-to-digital converter circuit 54 directly to boost DC-DC converter circuit 58. DC-DC converter provides an output voltage and current for ultrasonic actuator power switching circuit 22 through connector 59.

Digital-to-analog converter 54, optional envelope generator circuit 52 and DC-DC converter circuit 58 practically operate as a programmable power supply 100 controlled by microcontroller 10. The components that constitute programmable power supply 100 are selected to provide a settling time for the output voltage/current below 1 millisecond. Ultrasonic actuator power switching circuit 22 typically incorporates a pair of power MOSFET transistors complementarily clocked to provide an alternating current through connector 23 for the primary windings of a step-up transformer 24. The secondary winding of step-up transformer 24 is connected to a piezoelectric actuator 28 through connector 26. Piezoelectric actuator 28 located inside handpiece 34 is firmly attached to a nosecone 30 including a torsional feature 31 typically in the form of a spiral groove that can produce rotational oscillations at the tip of a lensectomy probe when driven at a suitable frequency. Nosecone 30 has a female thread at its distal end for firmly attaching and detaching a hollow lensectomy probe 32 having a tip 34 that can be bent and funnel shaped as shown in FIG. 1. Hollow lensectomy needle 32 is in fluid communication with an aspiration terminal 36 suitable for connection through flexible tubing to a source of vacuum typically located at the host lensectomy console. Connector 26 can provide an input signal for an actuator current detector circuit 39 and for an actuator voltage detector circuit 41. Detector circuits 39 and 41 provide output signals transmitted to microcontroller 10 through connectors 40 and 42 respectively.

### OPERATION

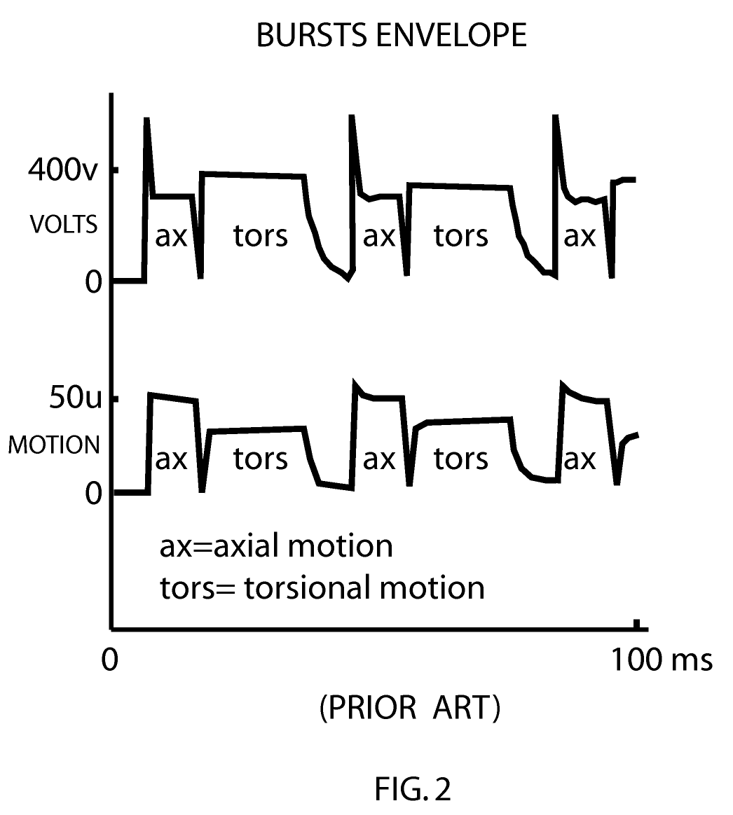

During operation, microcontroller 10 receives commands from external control 14 that transmit operator selected parameters such as amplitude and duration of the bursts of axial oscillations and also, amplitude and duration of the bursts of rotatory (lateral) oscillations. Typically an operator will activate a footpedal to operate the system. The host controller system will request to microcontroller 10 to deliver the corresponding ultrasonic burst pattern according to said footpedal status and console settings. Simultaneously the host controller can operate a vacuum source connected to connector 36. As depicted in the voltage and motion chart recordings of FIG. 2 a typical prior art system is configured to produce a driving signal for a piezoelectric actuator 28 aimed to obtain a burst of axial oscillations with a fast rise speed that levels at a preset motion amplitude. In the upper tracing of FIG. 2 the driving voltage for actuator 28 is shown. The lower tracing depicts the corresponding ultrasonic oscillations. It is clearly seen that for axial oscillations (AX), a 43 kHz frequency is provided at a driving voltage. This driving voltage produces rapid rise speeds, typically above 1.0 thousandths of an inch (mils) per millisecond of stroke.

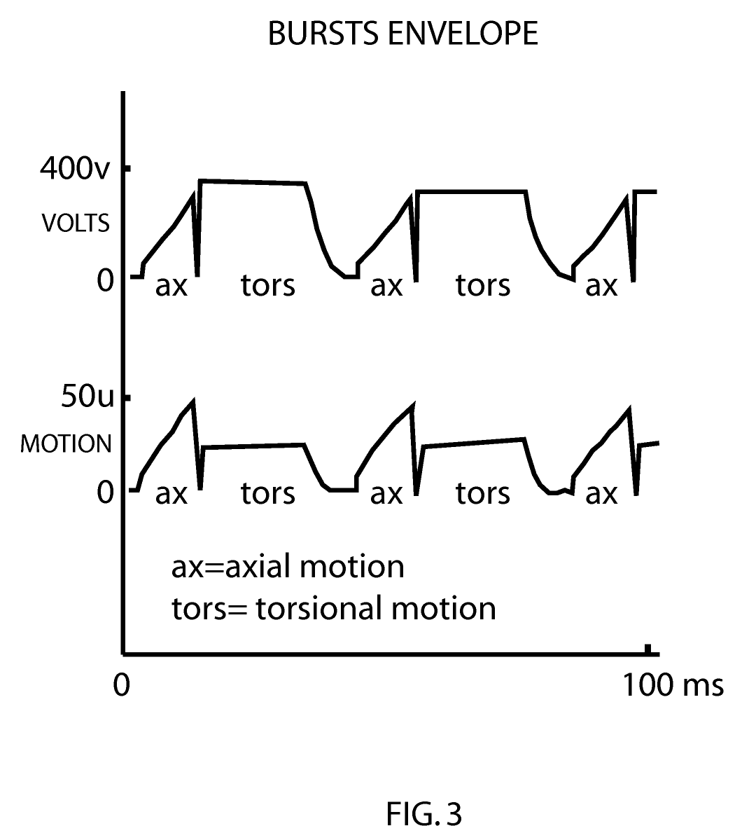

The fast rise speed produces the square shaped burst observed in the lower tracing for the axial component (AX). Feedback signals produced by current and voltage sensor circuits 39 and 41 are used by microprocessor 10 in a servo control modality to rapidly obtain and then keep steady the selected oscillation amplitude. Although advantageous, operating funnel shaped lensectomy probes in rotatory (torsional) mode has the problem of promoting clogging by cataract fragments. This undesirable situation is partially solved by programming the delivery of bursts of axial ultrasonic activity intercalated between the rotatory bursts. Bursts of axial ultrasonic activity are much more effective to avoid and resolve clogging at the funnel of lensectomy probes by cataract fragments because the jackhammer effect is applied to the fragments using different vectors. Intercalating bursts of axial ultrasonic power between the bursts of rotatory ultrasonic power in the way it is done in the prior art shown in FIG. 2 increases lens fragment repulsion from the tip of the lensectomy probe reducing efficiency and increasing turbulence. The method of the present invention consists in programming microprocessor 10 for the delivery of intercalated bursts of axial ultrasonic motion each having a slow rise speed, in opposition to the fast rise speed of the prior art axial bursts illustrated in FIG. 2. In the preferred embodiment of the present invention depicted in FIG. 3, microprocessor 10 commands clock generator 18 to produce resonant frequencies for piezoelectric actuator 28. These frequencies are typically predetermined during a tuning phase during priming of the system and can also be dynamically adjusted during operation using the current and voltage feedback signals from detectors 39 and 41. A typical twin-axial lensectomy handpiece has two main resonant frequencies. Using the same hardware, one frequency produces axial oscillations at the tip of a lensectomy probe 32 while a second resonant frequency produces rotatory oscillations. The shift between axial and rotatory motion of the lensectomy probe tip is produced by a particular configuration of the nosecone that produces two perpendicular axis of displacement depending on two different main resonant frequencies.

Currently available systems operate with an axial resonant frequency near 43 kHz and a rotational resonant frequency near 32 kHz. Microcontroller 10 under programmatic control can determine the production of bursts of axial oscillations instructing clock generator to deliver a clocking signal of about 43 kHz, and the production of bursts of rotational oscillations instructing clock generator to deliver a clocking signal of about 32 kHz. Using clock generator 18, microcontroller 10 can alternatively select dual-axis (torsional) oscillatory activity, axial oscillatory activity and an OFF state of the lensectomy probe. Actuator driver clock generator 18 can be an external circuit or a clock generator feature within microcontroller 10. During operation microcontroller 10 also has to determine the desired amplitude for the axial and the rotational oscillatory activities. In the prior art systems of FIG. 2 when the operator selects a “panel controlled mode” a fast settling and steady amplitude is produced at the tip of lensectomy probe 32 by microcontroller 10 command of analog-to-digital converter 54. The goal is to approximate as much as possible to deliver a constant stroke (power) according to operator power settings during the duration of the burst. Alternatively, when the operator selects a “surgeon controlled mode”, the digital signal provided to analog-to-digital converter 54 varies in proportion to the depression of a foot-pedal by the operation. The output voltage of converter 54 is fed to boost DC-DC converter 58 in a way the output voltage of power converter 28 can typically vary between 0 and 24 volts.

Operationally the combination of digital-to-analog circuit 54 and DC-DC converter 58 to constitute programmable power supply 100 under microcontroller 10 command that can be operated for envelope generation and consequently power modulation (amplitude modulation) of piezoelectric actuator 28. The output voltage/current of programmable power supply 100 is supplied to actuator power switching circuit 22. The power MOSFET transistors of switching circuit 22 are operated at the selected resonant frequency of actuator 28 in a way that a voltage, a current and a frequency suitable for the operation of actuator 28 is finally output at the secondary winding of transformer 24 and delivered across connector 26. The present invention incorporates ultrasonic burst envelope generator 52 operating under microcontroller command to reduce the rise speed of the axial bursts of ultrasonic oscillations. The preferred embodiment considers using microcontroller 10 features to obtain the envelope generator effect. In this embodiment of the present invention digital-to-analog converter 54 is dynamically operated by microcontroller 10 to produce the effect of a power modulator and of an envelope generator for the bursts of axial oscillations intercalated between the bursts of rotational oscillations. For each burst of axial ultrasonic oscillations, microcontroller 10 resets, adjusts and modulates the output power signal delivered to analog-to-digital converter 54 in a way that a selected amplitude modulation waveform if produced for each burst of axial oscillatory activity.

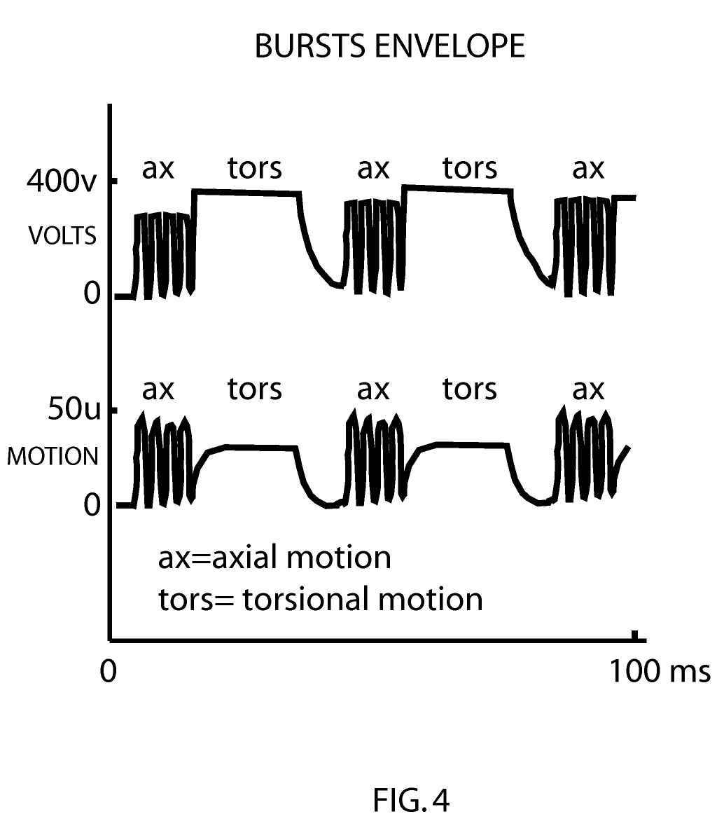

Providing a slow increase in stroke (amplitude) typically below 0.75 mils per millisecond, and until the operator selected power is achieved has demonstrated to be effective in significantly reducing chatter and repulsion. In the preferred embodiment, a ramp shaped stroke increase is preferred. Microcontroller 10 can programmatically use feedback signals from detectors 39 and 41 to dynamically adjust the output power signal delivered during each axial burst to converter 54 in a way that the desired attack waveform is obtained. Microcontroller 10 can make computations to adjust the attack waveform according to operator preferences and also according to other settings such as axial burst duration and amplitude. In a way of example, microcontroller 10 can provide an incremental signal to converter 54 in a way that the operator selected axial stroke is only achieved at the end of the axial burst duration (only attack and decay). Alternatively, microcontroller 10 can provide an incremental signal to converter 54 in a way that the operator selected axial stroke is achieved before the end of the axial burst duration and is then sustained until the end of the burst (attack, steady portion and decay). Microcontroller 10 can adjust the attack envelope to produce a proportional (ratio-metric) match for the selected stroke (amplitude) and/or for the selected burst duration (time). Microcontroller 10 can be programmed to deliver an attack portion of the axial bursts envelope with a shape that departs from a linear ramp such as exponential, logarithmic or other. An alternative embodiment for the operation of the present invention is shown in FIG. 4. Instead of providing a slow increase of stroke in a single burst of ultrasonic axial oscillations as shown in FIG. 3, in this embodiment microcontroller 10 commands the delivery of a train of axial bursts of equal amplitude during a selected interval intercalated between the rotational bursts of ultrasonic oscillations. The amplitude of each axial burst of the train corresponds to the operator selected axial amplitude (power). This mode of operation is achieved by programmatic activation and inactivation of clock generator 18 and/or analog-to-digital converter 54.

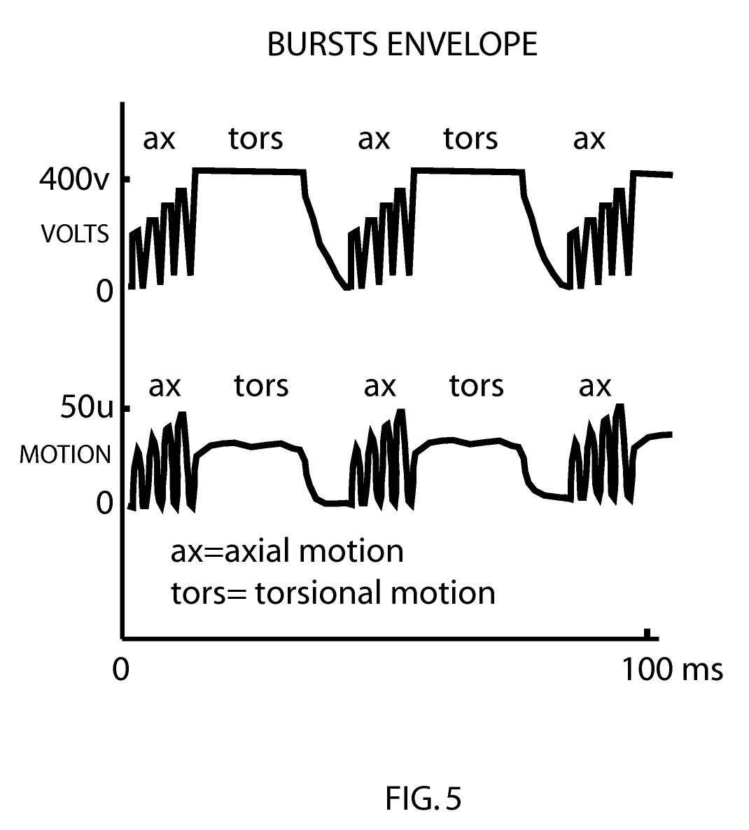

The attack portion of the envelope of each burst of axial oscillations of this embodiment can be modified to provide rise speeds below 0.75 mils/millisecond according to the embodiment depicted in FIG. 3. Another embodiment considers having microcontroller 10 to command the delivery of a train of axial bursts of different amplitudes during a selected interval intercalated between the rotational bursts of ultrasonic oscillations. The amplitude of each axial burst of the train can vary in several ways. As shown in FIG. 5 one mode of implementation of this embodiment considers a providing a sequential increment in the amplitude of individual bursts until reaching the operator selected axial amplitude (power). Similarly to the embodiment depicted in FIG. 3 the operator selected amplitude (power) can be achieved only the last burst of the train or alternatively sustained in more than one burst during the duration of the train. This mode of operation is achieved by parallel programmatic control of clock generator 18 and analog-to-digital converter 54 by microcontroller 10. In this embodiment microcontroller 10 controlled fluctuations of the amplitude of individual bursts of axial oscillations that compose a single train can depart from the linear increase of the embodiment shown in FIG. 5. For example, the maximum amplitude of the bursts of axial oscillations that compose a single train can increase and then decrease, can increase exponentially or logarithmically, can randomly vary without departing from the scope of the present invention.

By providing intercalated bursts of axial oscillatory activity with an attack portion of the envelope with a slow rising speed i.e. below 0.75 mils per millisecond, ultrasonic activity first seals the margins of the lens fragments allowing a vacuum to firmly hold the fragment for further axial ultrasonic activity of increasing amplitude (power). The same effect is obtained when a train of short axial pulses is delivered.

While the above description contains many specificities these should not be construed as limitations on the scope of the invention, but rather as an exemplification of preferred embodiments thereof. Many other variations are possible. For example the clock generator circuit can be a discrete programmable timer/counter circuit operated by microcontroller 10. The envelope generator circuit 52 composed for example by a constant current source disposed to repeatedly charge a capacitor can be installed at the output of analog-to-digital converter 54 to produce a steady rise speed of the attack portion of the burst of axial oscillations at a rate below 0.75 mils per millisecond. The same principle of operation of the present invention can be applied to the rotational component of the twin-axial lensectomy system. All these embodiments should be considered without departing from the present invention. Accordingly, the scope of the invention should be determined not by the embodiments illustrated but by the appended claims and their legal equivalents.

1. An anti-repulsion anti-clogging system for a dual-axis lensectomy handpiece comprising:
a. a microcontroller,
b. a dual axis operable lensectomy handpiece,
c. a programmable clock generator,
d. a programmable power supply,
whereby said microcontroller commands said programmable clock generator to operate said lensectomy handpiece to produce bursts of axial ultrasonic oscillatory activity intercalated with bursts of rotational ultrasonic oscillatory activity, and
whereby said microcontroller commands said programmable power supply in a way that the envelope of each of said bursts of axial oscillatory activity can be amplitude modulated.

2. The anti-repulsion anti-clogging system of claim 1 wherein the attack portion of said envelope of each of said bursts of axial oscillatory activity has a rise speed below 0.75 mils per millisecond.

3. The anti-repulsion anti-clogging system of claim 1 wherein the attack portion of said envelope of each of said bursts of axial oscillatory activity is ramp shaped.

4. An anti-repulsion anti-clogging system for operating a dual-axis lensectomy handpiece comprising:
a. a microcontroller,
b. a dual axis operable lensectomy handpiece,
c. a programmable clock generator,
d. a programmable power supply,
whereby said microcontroller commands said programmable clock generator to operate said lensectomy handpiece to produce bursts of axial ultrasonic oscillatory activity intercalated with bursts of ultrasonic rotational oscillatory activity, and
whereby said microcontroller commands said programmable power supply in a way that a train of bursts of axial ultrasonic oscillatory activity is intercalated between said bursts of rotational ultrasonic oscillatory activity.

5. The anti-repulsion anti-clogging system of claim 4 wherein said microcontroller programs said programmable power supply to deliver each of said bursts of axial oscillatory activity composing said train at about the same amplitude.

6. The anti-repulsion anti-clogging system of claim 4 wherein said microcontroller programs said programmable power supply to deliver each of said bursts of axial oscillatory activity composing said train at different amplitudes.

7. The bursts of axial oscillatory activity composing said train of claim 6 wherein said microcontroller programs said programmable power supply to deliver each of said bursts of axial oscillatory activity composing said train in a sequence with increasing amplitude.

8. A method to reduce repulsion and chatter of lens fragments at the tip of a lensectomy probe while operating a dual axis ultrasonic lensectomy handpiece comprising the steps of:
a. providing a microcontroller,
b. providing a dual axis operable lensectomy handpiece,
c. providing a programmable clock generator,
d. providing a programmable power supply,
e. programming said microcontroller to command said clock generator to deliver bursts of axial ultrasonic oscillatory activity intercalated with bursts of rotational ultrasonic oscillatory activity,
e. programming said microcontroller to command said programmable power supply to modulate the amplitude of the envelope of said bursts of axial ultrasonic oscillatory activity.

9. The method of claim 8 whereby said modulation of the amplitude of said bursts of axial ultrasonic oscillatory activity consists in a reduction of the rise speed of the attack portion of said envelope to a value below 0.75 mils per millisecond.

10. The method of claim 8 whereby said modulation of the amplitude of said bursts of axial ultrasonic oscillatory activity consists in a segmentation of said burst into a plurality of bursts to produce a train of bursts of equal amplitude.

11. The method of claim 8 whereby said modulation of the amplitude of said bursts of axial ultrasonic oscillatory activity consists in a segmentation of said burst into a plurality of bursts to produce a train of bursts of increasing amplitude.
